(19) United States
(12) Patent Application Publication
(10) Pub. No.: US 2007/0231359 A1
(43) Pub. Date: Oct. 4, 2007

(54) Methods and compositions for piercing studs with bioactive polymer coating

(76) Inventors: Daniel James Murauski, Naperville, IL (US); Jonas Moses, Chicago, IL (US)

Correspondence Address: Daniel Murauski, 2326 Riverwoods Dr., Naperville, IL, 60565, US

(21) Appl. No.: 11/397,473

(22) Filed: Apr. 3, 2006

(51) Int. Cl. A61K 38/14; A61K 31/7048; A61K 31/7034; A61K 31/65; A61K 31/63; A61K 33/38

(52) U.S. Cl. 424/422; 424/618; 514/8; 514/35; 514/152; 514/155; 514/28

(57) ABSTRACT

Devices and methods for developing piercing instruments and studs that have bioactive polymer coating to promote healing, to minimize post-piercing infection, and to reduce inflammation are disclosed.

### FIELD OF THE INVENTION

This disclosure generally relates to devices and methods for developing piercing instruments and studs that have bioactive polymer coating to promote healing and to minimize post-piercing infection.

### BACKGROUND

Infection at the site of piercing is common. In addition, the healing time for piercing studs varies depending on the nature and kind of studs and the site of piercing. For example, the healing time for a stud pierced in the earlobes, eyebrows, nose, and lips is about 6 to 8 weeks; for ear cartilage, about 6 to 12 weeks; for inside of the mouth or the tongue, about 3 to 6 weeks; for nipple, about 2 to 4 months; for genitals, about 9 months; for navel, about 12 months. Extended healing time is a discomfort to the user and increases chance of infection.

It is desirable to have a coating that, when applied to the piercing instrument and the piercing stud, minimizes skin-metal irritation, reduces the healing time, and minimizes the risk of infection.

### SUMMARY

Methods and compositions to minimize infection, to promote healing, and to reduce inflammation for a piercing stud are disclosed. Bioactive coatings disclosed herein can be applied to any piercing stud.

### DETAILED DESCRIPTION

While the present disclosure may be susceptible to embodiments in different forms, the embodiments described in detail herein are to be considered exemplifications of the principles of the disclosure and are not intended to be exhaustive or to limit the disclosure to the details of construction and the arrangements of components set forth in the following description.

Bioactive coatings disclosed herein can be sprayed on the piercing stud to prevent metal-skin irritation. Bioactive coatings disclosed herein can also be pre-applied during the manufacture of the studs. Although the coatings may not aid in healing during the initial piercing procedure, they promote healing during later periods.

The bioactive coatings can also be sprayed after the piercing stud has been removed to minimize any allergy the user may develop to the coating during the initial piercing procedure. In this scenario, the coating acts as more of an insulating layer for the metal-skin contact surface.

The bioactive coatings aid the healing process and help to reduce the immune response, including undesirable inflammatory reactions, by reducing infections.

Piercing studs seeded or coated with bioactive material to maintain a bacteriostatic environment will not only promote a bacteria-free environment; they will also aid in the healing process of a puncture wound caused by the piercing procedure.

Common materials for piercing studs comprise of gold, silver, stainless steel or any other biologically inert metals. Bioactive coatings disclosed herein can minimize local skin irritation, diminish infections, reduce inflammatory immune response, and promote healing.

Piercing studs for face (earlobes, nose, eyebrows, and lips), ear cartilage, interior of the mouth or tongue, nipple, genital, and navel contain a bioactive coating disclosed herein. Piercing studs made from surgical stainless steel, titanium or biocompatible polymers will be coated with a polymer or copolymer seeded or impregnated with bioactive molecules. Optionally, the coating may degrade over a period of time leaving behind a normal piercing stud. The coating may also stay on the piercing and the stud may be replaced after the piercing site has attained a desired healing state. The bioactive coating functions to increase the rate of healing and reduce the risk of infection at the piercing site. Swelling or inflammation at the piercing site will also decrease because of the lowered immune response.

Suitable bioactive materials that can be applied as a polymer coating to piercing studs include antibiotic/bacteriostatic agents, such as, for example macrolides, tetracyclines, or sulphonamides; and non-steroidal anti-inflammatory agents, such as, for example, Voltaren™ or Acular™.

In an aspect, a high percentage of the bioactive molecule reaches the immediate tissue target without a substantial escape of the bioactive molecules into circulation or surrounding tissue and, preferably, the delivery of the device with the drug to the site is atraumatic and does not contribute to or cause additional medical problems or risk.

Bioactive coated piercing studs also minimize infections caused by mishandling of the stud and also from the self-induced infection from the subject's own microflora.

A suitable polymer coating impregnated with necessary bioactive molecules is capable of delivering the drugs in an efficient, sustained fashion with minimal local irritation and any other adverse reactions. The bioactive polymer can suitably be applied onto the device such as a piercing stud prior to insertion at a target site or can be pre-coated during manufacture of the stud. The concentration of the active ingredient of the drug and the polymer percentage will depend on the nature of the piercing stud and the target site.

In addition to antimicrobial and anti-infectious compounds, skin nutrients to promote healing may also be supplemented in the bioactive polymer coating. Agents that minimize scarring can also be included if necessary. These antimicrobial compounds and other agents can also be formulated in a sustained release formulation to insure a uniform release over an extended period of time. For example, the sustained release formulation can be designed so that the delivery of the drug is maximized when the potential infection is usually known to occur (for example, one week to ten days after piercing or any other suitable time period). The necessary drugs or compounds can also be impregnated or imbibed into the polymer coating whenever necessary. For environmentally sensitive drugs, this may be an option.

A suitable polymer used herein is durable, poses minimal skin or bodily irritation, is capable of holding the various antimicrobial compounds and other nutrients, can be coated onto a metallic surface such as gold, silver, or stainless steel, and remains stable over a longer period of time. It is also within the scope of this disclosure to formulate polymer or copolymers that disintegrate over a period of time to coincide with the healing of the target site wherein further chance for a fresh infection is minimal. The bioactive polymer coating of the present disclosure overcomes several of the aforementioned limitations. The sustained delivery of the anti-microbial compounds from the bioactive polymer provides an aseptic local environment and the various skin nutrients promote healing if necessary. Polymer substrates that can be treated with the technique include polymers and co-polymers of polyurethanes, polyolefins, polyesters, PVCs, polyamines, polyimines, and silicone. Any other polymer coating that is biocompatible, lubricious, antimicrobial, and that can be applied to piercing studs is within the scope of this invention.

Materials and Methods

1. Polymer Base

A polymer coating suitable for coating piercing studs, RepelaCOAT™, can be obtained from Advanced Surface Technology Products (Billerica, Mass.), which is crosslinked and covalently bound to the substrate. In this coating, silver salts and/or antibiotics are ionically bonded to the supporting polymer. In the presence of bodily cations such as sodium or calcium, the anti-microbial agents are ionically exchanged with these physiological cations. Long chain hydrophilic polymers adsorb water molecules and facilitate the ion exchange process. RepelaCOAT™ sustained release formulation prevents bacterial adherence by it's slow ion-exchange release of anti-microbial agents.

Another suitable polymer coating, Polycil™, can be obtained from BioInteractions Ltd (Reading, England). Polycil™ is an anti-microbial coating to combine non-thrombogenic component with an anti-microbial component. In Polycil™, polyhexanide, the anti-microbial component, is functionalized with a methacrylate group and co-polymerised with methoxy polyethylene glycol (2000) methacrylate (MPEG2000MA), methoxy polyethylene glycol (350) methacrylate (MPEG350MA) and butyl methacrylate.

Other suitable polymer coatings can be prepared as described in Hahn et al (2004), “Anti-inflammatory drug delivery from hyaluronic acid hydrogels,” Journal of Biomaterial Science (in publication), which describes two types of hyaluronic acid (HA) hydrogels synthesized by crosslinking HA with divinyl sulfone (DVS) and poly(ethylene glycol)-divinyl sulfone (VS-PEG-VS). Markvicheva et al (2002), “Polymer coatings with immobilized thrombin and peptides: preparation and use for wound healing” (in Russian), Vopr. Med. Khim., describes polymer dressings, with encapsulated thrombin or synthetic peptides that can mimic thrombin action, that are employed for wound healing. Markvicheva et al describes the method for preparation of these hydrogel composites of PVCL-CaAlg [poly(N-vinyl caprolactam-calcium alginate). Nablo et al (2004), “Nitric oxide-releasing sol-gels as antibacterial coatings for orthopedic implants”, Journal of Biomaterials, describes nitric oxide (NO)-releasing sol-gels as potential antibacterial coatings for orthopedic devices: medical-grade stainless steel is coated with a sol-gel film of 40% N-aminohexyl-N-aminopropyltrimethoxysilane and 60% isobutyltrimethoxysilane. Blakera et al (2003), “Development and characterisation of silver-doped bioactive glass-coated sutures for tissue engineering and wound healing applications,” Journal of Biomaterials, describes a silver-doped bioactive glass powder (AgBG) to coat resorbable Vicryl® (polyglactin 910) and non-resorbable Mersilk® surgical sutures. Stable and homogeneous coatings on the surface of the sutures were achieved using an optimised aqueous slurry-dipping technique. Changez et al (2003), “The effect of composition of poly(acrylic acid)-gelatin hydrogel on gentamicin sulphate release: in vitro,” Journal of Biomaterials, describes hydrogels based on poly(acrylic acid) and gelatin crosslinked with N,N′-methylene bisacrylamide (0.5 mol %) and glutaraldehyde (4%).

2. Coating Methods

The polymer or copolymer along with the suitable ingredients such as antimicrobials and other nutrients, if any, is coated onto the surface of the stud using techniques known to a person of ordinary skill in the art. For example, aqueous slurry dipping of the polymer along with the anti-microbial and/or healing agents is a suitable method to coat the piercing studs.

We claim:

1. A method of minimizing infection for a piercing stud, the method comprising:
(a) obtaining a piercing stud; and
(b) applying a bioactive coating that reduces infection at a piercing site.

2. A method of reducing healing time for a piercing stud, the method comprising:
(a) obtaining a piercing stud; and
(b) applying a bioactive coating that reduces healing time at a piercing site.

3. A method of reducing inflammation for a piercing stud, the method comprising:
(a) obtaining a piercing stud; and
(b) applying a bioactive coating that reduces healing time at a piercing site.

4. The method of claim 1, wherein the bioactive coating comprises at least one anti-microbial compound.

5. The method of claim 2, wherein the bioactive coating comprises at least one healing compound.

6. The method of claim 1 or 2 or 3, wherein the bioactive coating is applied prior to inserting the piercing stud at a desired location.

7. The method of claim 1 or 2 or 3, wherein the bioactive coating is applied during the manufacture of the piercing stud.

8. The method of claim 3, wherein the anti-microbial compound is selected from the group consisting of macrolides, tetracyclines, sulphonamides, clindamycin, vancomysin, telcoplanin, cefotaxime, chlorohexidine, silver sulphadiazine, silver salts and iodine.

9. The method of claim 1 further comprising an anti-inflammatory agent to reduce local skin irritation.

10. The method of claim 1 or 2, wherein the bioactive coating comprises a polymer formulation capable of delivering a desired drug or a compound over a sustained period of time.

11. The method of claim 1 or 2, wherein the bioactive coating is biocompatible.

12. A piercing stud comprising a bioactive coating capable of reducing infection at a desired target site and capable of reducing healing time.

13. A piercing stud comprising a bioactive coating capable of reducing inflammation at a desired target site and capable of reducing healing time.
